(19) United States
(12) Patent Application Publication
(10) Pub. No.: US 2012/0184713 A1
(43) Pub. Date: Jul. 19, 2012

(54) Compositions and Methods for Modulation of Bone Density and Biomineralization

(76) Inventors: Kenneth D. Irvine, Highland Park, NJ (US); Hiroyuki O. Ishikawa, Tokyo (JP)

(21) Appl. No.: 13/353,717

(22) Filed: Jan. 19, 2012

Related U.S. Application Data
(60) Provisional application No. 61/434,087, filed on Jan. 19, 2011.

(51) Int. Cl. C07K 14/435; C07C 57/00; G01N 33/573; C07H 21/02; C12Q 1/48; C08B 37/00; C07H 21/04

(52) U.S. Cl. 530/350; 536/123.1; 554/1; 536/23.1; 536/24.5; 435/15; 435/7.1

(57) ABSTRACT

Compositions and methods for modulating FAM20C kinase action and identifying therapeutic agents for the same are disclosed.

This application claims priority to U.S. Provisional Application No. 61/434,087 filed Jan. 19, 2011, the entire contents being incorporated herein by reference as though set forth in full.

### FIELD OF THE INVENTION

This invention relates to the fields of protein biochemistry and signal transduction. More specifically, the invention provides compositions and methods for identifying agents which modulate family with sequence similarity 20 (FAM20) protein activity, and the use of such agents for the treatment of bone density disorders and certain types of dysplasia.

### BACKGROUND OF THE INVENTION

Several publications and patent documents are cited throughout the specification in order to describe the state of the art to which this invention pertains. Each of these citations are incorporated herein by reference as though set forth in full.

Protein phosphorylation is mediated by enzymes called protein kinases. Phosphorylation is one of the most common forms of post-translational modification for cytoplasmic and nuclear proteins. Because cell signaling and metabolic processes are frequently regulated by protein kinases, they have been attractive targets for drug development. Indeed, small molecules that inhibit the activity of particular classes of protein kinases have become important anti-cancer drugs (Ventura, J. J. et al., (2006) Clin Transl Onco18:153-160.

However, phosphorylation of secreted proteins is rare, and with one recent exception (Ishikawa, H. O., et al. (2008) Science 321: 401-404), kinases that phosphorylate proteins or protein domains destined to be extracellular have not been identified. All existing kinases inhibitors target cytoplasmic kinases.

One class of secreted proteins that are known to be phosphorylated are the SIBLINGs (Small Integrin-Binding LIgand, N-linked Glycoprotein) (Fisher, L. W., et al., (2003) Connective tissue research 44 Suppl 1, 33-40). These include Osteopontin (OPN), Bone Sialoprotein 2 (BSP2), Dentin Matrix Protein 1 (DMP1), Dentin Matrix Protein 3 (DMP3), and Matrix Extracellular Phosphoglycoprotein (MEPE). The SIBLINGs were first identified as proteins expressed in bone and teeth, although they are actually expressed more broadly. Bone and teeth are also composed of a calcium-phosphate-based mineral (hydroxyapatite). Multiple roles for SIBLING proteins in bone and teeth have been proposed, including both structural and regulatory roles (e.g., by modulating the formation of hydroxyapatite). SIBLING proteins can influence biomineralization, and this influence can depend on their phosphorylation state (Qin, C. et al., (2004) Crit. Rev Oral Biol Med 15:126-136; Qin, C. et al., (2007) Journal of Dental Research 86: 1134-1141). Analysis of their biological functions in vivo is complicated by the potential for some redundancy among them, but DMP1 mutations in humans have been linked to Hypo-phosphatemic Rickets (Feng, J. Q., et al., (2006) Nature Genetics 38:1310-1315), and DMP1 mutation in mice resulted in a hypomineralized bone phenotype (Ye, L., et al., (2004) J. Biol. Chem. 279: 19141-19148). OPN generally acts as an inhibitor of biomineralization (Steitz, S. A. et al., (2002) American Journal of Pathology 161: 2035-2046).

There has been substantial interest in SIBLING proteins recently because elevated expression of SIBLINGs, especially OPN, has been linked to several types of cancer (Bellahcene, A. et al., (2008) Nature Reviews 8: 212-226; Johnston, N. I., et al., (2008) Front Biosci 13: 4361-4372). SIBLING proteins have been implicated in multiple stages of cancer progression, but clinical interest in OPN has focused on the observation that expression of OPN is highly correlated with metastasis, and recent studies in mice demonstrated critical functional roles for OPN in promoting metastasis. Although the role of SIBLING protein phosphorylation has not been studied in terms of its potential influence on cancer, it is thought in general that phosphorylation plays important roles in modulating the activity of SIBLING proteins, in which case inhibition of OPN phosphorylation might prevent it from promoting metastasis.

FAM20 (Family with sequence similarity 20) proteins were initially identified as a conserved family of three related proteins (FAM20A, FAM20B, FAM20C). More recently, a human genetic disease, Raine Syndrome (lethal osteosclerotic bone dysplasia), was linked to FAM20C [18]. Publications on FAM20 proteins describe their biochemical function as “unknown,” and have reported that FAM20 proteins are secreted (Nalbant, D. et al., (2005) BMC genomics 6: 11).

Inasmuch as mutations in FAM20 protein have been correlated with bone dysplasia, it would be highly desirable to further characterize the biochemical functions of this protein in order to identify agents which modulate such activity. Such agents should have utility for the treatment and prevention of bone dysplasia, osteoporosis and cancer.

### SUMMARY OF THE INVENTION

The present inventors have discovered that FAM20C acts as a protein kinase that modulates cellular processes involved in bone and teeth formation. Agents which modulate this kinase activity should have efficacy for the treatment of bone disorders such as dysplasia and osteoporosis. Thus, in accordance with the present invention, a method for identifying an agent that modulates FAM20C protein kinase activity is provided. An exemplary method entails incubating FAM20C protein and at least one substrate under conditions effective for kinase action to occur, in the presence and absence of the agent to be tested; and assessing the phosphorylation level of said substrate, agents which alter the phosphorylation level of said substrate relative to FAM20C kinase incubated in the absence of said agent being effective to modulate FAM20C kinase activity. Such assays may be performed in vitro and in vivo. In a preferred embodiment, the substrate is a SIBLING protein or a phosphorylatable fragment thereof. Agents so identified may also be assessed in vivo animal models of bone formation and biomineralization processes.

### BRIEF DESCRIPTION OF THE DRAWINGS

FIG. 1. Sequence similarity among Golgi kinases. Alignment of Fj and FAM20 proteins from diverse species. Amino acid similarities are highlighted by coloring, Orange bars at top highlight amino acids common to all kinases, bars at bottom indicate relative conservation of specific amino acids.

FIG. 2. FAM20C is a Golgi protein. a) Relative similarity among Golgi kinases in flies and humans. B) Western blot showing that FAM20C:V5 expressed in cultured cells is detected in both lysate (L) and medium (M). c) Localization of FAM20C:V5 (green) in 293 cells overlaps a Golgi marker (Giantin, magenta). d) FAM20C:V5 localization is distinct from an ER marker (anti-KDEL, magenta). Panels marked by prime symbols show single channels of the stain to the left.

FIG. 3. Localization of FAM20 proteins. a-h Localization of the indicated V5-tagged FAM20 proteins (green) transfected into 293 cells (a-d) or S2 cells (e-h) as compared to Golgi markers (Giantin, a,c, or p120 Golgi, e,g, magenta) or an ER marker (anti-KDEL, magenta). Panels marked by prime symbols show single channels of the stain to the left. i) CG31145:V5 expressed in S2 cells is detected in both lysate (L) and medium (M), CG3631:V5 is only detected in lysate.

FIG. 4. Kinase activity of FAM20C a) Autoradigram on protein gel showing the results of in vitro kinase assays in the presence (+) or absence (−) of FAM20C:V5 and the proteins indicated at bottom as substrates; the mobilities of FAM20C and Casein are indicated. b) Time course of FAM20C kinase reaction using dephosphorylated alpha Casein as a substrate. c) Relationship between kinase activity and amount of wild-type or D443G mutant FAM20C:V5. D) Relationship between kinase activity and amount of Casein, curve fitting was used to determine Km.

FIG. 5. Kinase activity of FAM20C All panels show the results of kinase assays using dephosphorylated alpha casein as a substrate, and affinity purified FAM20C:V5 as the enzyme. a) Relationship between kinase activity and amount of ATP, curve fitting was used to determine Km. b) Dependency of kinase activity on divalent cation concentration. c) Dependency of kinase activity on pH. d) Dependency of kinase activity on calcium concentration.

FIG. 6. FAM20C phosphorylates SIBLING proteins a) Western blot (anti-FLAG) showing mobility shift of Opn:FLAG induced by co-transfection of FAM20C:V5 (as indicated by +) in 293 cells, and its reversal by phosphatase. b) Western blots (anti-FLAG) showing mobility shifts of Bsp:FLAG and MEPE:FLAG induced by co-transfection of FAM20C:V5, but not FAM20A:V5 (as indicated by +), in 293 cells. c) Autoradigram on protein gel showing the results of in vitro kinase assays in the presence (+) or absence (−) of FAM20C:V5 and the proteins indicated at bottom as substrates; the mobility of FAM20C is indicated. d) Sequence of peptides used for kinase assays. First three amino acids (gray) were added to enable assays using phosphoceulllose paper. 1. OPN-ASARM peptide, Ser residues are highlighted in blue, G-CK consensus sites are underlined. In peptides 2-4, combinations of Ser residues were mutated to Ala. e) Results of kinase assays using the indicated peptides (—indicates no peptide), using the indicated kinases.

FIG. 7. Sequence of FAM20C and mutant isoforms a) Amino acid sequence of human FAM20C(NP_{—}064608.2). Amino acids altered in human patients or by our site-specific mutagenesis are identified by colors (red, from patients, blue, in conserved motifs) and numbers above. b) Tabulation of the location of mutated amino acids, to account for differences in annotation due to different sequence entries of human FAM20C, and differences in size of human versus mouse FAM20C.

FIG. 8. Localization and activity of FAM20C mutant isoforms a-d Examples of localization of the indicated V5-tagged FAM20C mutant proteins (green) transfected into 293 cells, as compared to a Golgi markers (Giantin, a,c, red) or an ER marker (anti-KDEL, b,d red). Panels marked by prime symbols show single channels of the stain to the left. e) Western blot showing localization of FAM20C mutant proteins in the lysate (L) and/or medium (M) of 293 cells. f) Results of kinase assays using the indicated FAM20C mutant proteins and the OPN-ASARM peptide (1) as a substrate.

FIG. 9. Localization of FAM20C mutant isoforms Examples of localization of the indicated V5-tagged FAM20C mutant proteins (green) transfected into 293 cells, as compared to a Golgi markers (Giantin, a-h, red) or an ER marker (anti-KDEL, i-p, red).

### DETAILED DESCRIPTION OF THE INVENTION

The main structural component of bone (osseous tissue) is a composite of secreted extracellular proteins and the mineral hydroxyapatite. Insufficient bone density is a significant health concern for a majority of the human population as they age. Excess mineralization is also implicated in pathological conditions, including atherosclerosis. Human genetic diseases can identify proteins that modulate biomineralization. Raine syndrome (lethal osteosclerotic bone dysplasia) is associated with increased ossificiation resulting in skeletal malformation¹⁻³. Raine syndrome is caused by mutations in FAM20C⁴⁻⁶, which has been reported to encode a secreted component of bone and teeth⁷.

Here we show that FAM20C encodes a novel, Golgi-localized protein kinase. FAM20C can phosphorylate known secreted phosphoproteins, and characterization of its activity identifies FAM20C as a Golgi casein kinase. FAM20C substrates include phosphoproteins and peptides with known roles in regulating biomineralization, including Osteopontin and other members of the SIBLING family. Introduction of point mutations identified in human patients into recombinant FAM20C impairs its normal cellular localization and kinase activity.

Our results establish the biochemical basis for Raine syndrome, identify FAM20C as a kinase for secreted phosphoproteins, and provide in vivo, genetic confirmation of the importance of secreted protein phosphorylation to the regulation of biomineralization. FAM20C mutations identified in human patients result in increased bone mass. We demonstrate that certain of these mutations impair FAM20C kinase actitivity. Accordingly, FAM20C inhibitory agents should be effective to increase bone mass and thus be effective for the treatment of disorders such as osteoporosis. Our data also suggest that compounds that modulate FAM20C kinase activity could be effective for modulating bone density and biomineralization.

### DEFINITIONS

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds.

According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

A “single nucleotide polymorphism (SNP)” refers to a change in which a single base in the DNA differs from the usual base at that position. These single base changes are called SNPs or “snips.” Millions of SNP's have been cataloged in the human genome. Some SNPs such as that which causes sickle cell are responsible for disease. Other SNPs are normal variations in the genome.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

The term “biomineralization” refers to the formation or accumulation of minerals by organisms especially into biological tissues or structures such as bones, teeth, and shells.

The phrase “bone dysplasia” refers to a condition characterized by abnormal bone growth, more frequently occurring in children. There are a great many varieties of bone dysplasia, many of which are caused by genetic disorders (e.g., mutations in the FAM20C gene), or by disturbances in the levels of growth hormones in the blood. They are also often referred to as skeletal dysplasias. Sometimes these growth disorders may lead to other problems such as limb deformities that make movement difficult, and spinal deformities, such as scoliosis.

The term “osteoporosis” refers to is a disease of bones that leads to an increased risk of fracture. Osteoporosis literally means ‘porous bones’. In osteoporosis the bone mineral density (BMD) is reduced, bone microarchitecture is deteriorating, and the amount and variety of proteins in bone is altered.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

“Sample” or “patient sample” or “biological sample” generally refers to a sample which may be tested for a particular molecule. Samples may include but are not limited to cells, body fluids, including blood, serum, plasma, bone aspirate, urine, saliva, tears, CSF, pleural fluid and the like.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

“Target nucleic acid” as used herein refers to a previously defined region of a nucleic acid present in a complex nucleic acid mixture wherein the defined wild-type region contains at least one known nucleotide variation which may or may not be associated with a bone density disorder. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased. It is also advantageous for some purposes that a nucleotide sequence be in purified form.

The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated. Thus, the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5° C.+16.6 Log[Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5× Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide” or “oligo” as used herein means a short sequence of DNA or DNA derivatives typically 8 to 35 nucleotides in length, primers, or probes. An oligonucleotide can be derived synthetically, by cloning or by amplification. An oligo is defined as a nucleic acid molecule comprised of two or more ribo- or deoxyribonucleotides, preferably more than three. The exact size of the oligonucleotide will depend on various factors and on the particular application and use of the oligonucleotide. The term “derivative” is intended to include any of the above described variants when comprising an additional chemical moiety not normally a part of these molecules. These chemical moieties can have varying purposes including, improving solubility, absorption, biological half life, decreasing toxicity and eliminating or decreasing undesirable side effects.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

An “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease III cleavage from longer dsRNA and are the mediators of sequence-specific mRNA degradation. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting FAM20C RNA may be between 15-35 nucleotides in length, and more typically about 21 nucleotides in length.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, peptide-tethering, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the FAM20C nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the FAM20C encoding nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, small molecules, antibodies, peptides, peptide/DNA complexes, and any nucleic acid based molecule, for example an oligo, which exhibits the capacity to modulate the activity of the FAM20C nucleic acids described herein or their encoded proteins. Agents are evaluated for potential biological activity by inclusion in screening assays described herein below.

The term “modulate” as used herein refers increasing or decreasing. For example, the term modulate refers to the ability of a compound or test agent to interfere with any biological activity of the FAM20C protein, particularly the kinase activity.

A kinase inhibitor inhibits the attachment of phosphate molecules to phosphorylation sites on target protein molecules. Kinase inhibitors are commercially available and several are in clinical trials.

### Methods of using Fam20C Encoding Nucleic Acids and Proteins for Detection of Agents Useful for Modulating Bone Density and Biomineralization

FAM20C containing nucleic acids may be used for a variety of purposes in accordance with the present invention. FAM20C DNA, RNA, or fragments thereof may be used as probes to detect the presence of and/or expression of these molecules. Methods in which nucleic acids may be utilized as probes for such assays include, but are not limited to: (1) in situ hybridization; (2) Southern hybridization (3) northern hybridization; and (4) assorted amplification reactions such as polymerase chain reactions (PCR).

Assays for detecting FAM20C genetic alterations associated with disease may be conducted on any type of biological sample, including but not limited to body fluids (including blood, urine, serum, cerebral spinal fluid, bone aspirates), any type of cell (such as white blood cells, mononuclear cells bone cells or bone cell progenitors) or body tissue. In most embodiments for screening for nucleic acids containing FAM20C alterations, the nucleic acid in the sample will initially be amplified, e.g. using PCR, to increase the amount of the template as compared to other sequences present in the sample. This allows the target sequences to be detected with a high degree of sensitivity if they are present in the sample. This initial step may be avoided by using highly sensitive array techniques that are becoming increasingly important in the art. Alternatively, new detection technologies can overcome this limitation and enable analysis of small samples containing as little as 1 μg of total RNA. Using Resonance Light Scattering (RLS) technology, as opposed to traditional fluorescence techniques, multiple reads can detect low quantities of mRNAs using biotin labeled hybridized targets and anti-biotin antibodies. Another alternative to PCR amplification involves planar wave guide technology (PWG) to increase signal-to-noise ratios and reduce background interference. Both techniques are commercially available from Qiagen Inc. (USA).

Since the alterations in FAM20C have been associated with bone disorders, methods for identifying agents that modulate the activity of the genes and their encoded products should result in the generation of efficacious therapeutic agents for the treatment of a variety of disorders associated with this condition.

Molecular modeling should facilitate the identification of specific organic molecules with capacity to bind to the active site of the proteins encoded by FAM20C nucleic acids based on conformation or key amino acid residues required for function. A combinatorial chemistry approach will be used to identify molecules with greatest activity and then iterations of these molecules will be developed for further cycles of screening.

The polypeptides or fragments employed in drug screening assays may either be free in solution, affixed to a solid support or within a cell. In one approach, inhibitors will be tested in vitro for kinase inhibitory activity, preferably in high throughput format. Another method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered FAM20C gene. These host cell lines or cells are defective at the polypeptide level. The host cell lines or cells are grown in the presence of drug compound. The ability of the compound to modulate bone formation, assembly or phosphorylation associated processes is then measured to determine if the compound is capable of modulating such processes in the defective cells. Host cells contemplated for use in the present invention include but are not limited to bacterial cells, fungal cells, insect cells, mammalian cells, and bone cells. The FAM20C encoding DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

Cells and cell lines suitable for studying the effects of the FAM20C directed agents on phosphorlation of relevant targets and methods of use thereof for drug discovery are provided. Such cells and cell lines will be transfected with the FAM20C encoding nucleic acids described herein and the effects on kinase activity, biomineralization and bone denisity can be determined. Such cells and cell lines could also be contacted with siRNA molecules to assess the effects thereof.

A wide variety of expression vectors are available that can be modified to express the novel DNA or RNA sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1/V5&His (Invitrogen); baculovirus vectors such as pVL1392, pVL1393, or pAC360 (Invitrogen); and yeast vectors such as YRP17, YIPS, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the gal4 inducible promoter and the PGK constitutive promoter.

In addition, a vector of this invention may contain any one of a number of various markers facilitating the selection of a transformed host cell. Such markers include genes associated with temperature sensitivity, drug resistance, or enzymes associated with phenotypic characteristics of the host organisms.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacophore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacophore.

Thus, one may design drugs which have, e.g., improved polypeptide activity or stability or which act as inhibitors, agonists, antagonists, etc. of polypeptide activity. By virtue of the availability of FAM20C nucleic acid sequences described herein, sufficient amounts of the encoded polypeptide may be made available to perform such analytical studies as x-ray crystallography. In addition, the knowledge of the protein sequence provided herein will guide those employing computer modeling techniques in place of, or in addition to x-ray crystallography.

In another embodiment, the availability of wild type and altered FAM20C encoding nucleic acids enables the production of strains of laboratory mice carrying such altered and wild type molecules. Transgenic mice expressing these nucleic acids provide a model system in which to examine the role of the FAM20C protein in the development and progression of bone disorders. Methods of introducing transgenes in laboratory mice are known to those of skill in the art. Three common methods include: (1) integration of retroviral vectors encoding the foreign gene of interest into an early embryo; (2) injection of DNA into the pronucleus of a newly fertilized egg; and (3) the incorporation of genetically manipulated embryonic stem cells into an early embryo. Production of the transgenic mice described above will facilitate the molecular elucidation of the role that FAM20C protein plays in various cellular metabolic processes, including: phosphorylation of biologically relevant targets and bone mineralization. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extra-chromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated FAM20C genes as insertional cassettes to selectively inactivate a wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extra-chromosomal recombination, which occurs at a frequency approaching 100%, homologous plasmid-chromosome recombination was originally reported to only be detected at frequencies between 10⁻⁶ and 10⁻³. Non-homologous plasmid-chromosome interactions are more frequent occurring at levels 10⁵-fold to 10² fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodou-racil, (FIAU). By this counter selection, the number of homologous recombinants in the surviving transformants can be increased. Utilizing FAM20C containing nucleic acid as a targeted insertional cassette provides means to detect a successful insertion as visualized, for example, by acquisition of immunoreactivity to an antibody immunologically specific for the FAM20C polypeptide and, therefore, facilitates screening/selection of ES cells with the desired genotype.

As used herein, a knock-in animal is one in which the endogenous murine gene, for example, has been replaced with the human FAM20C gene of the invention. Such knock-in animals provide an ideal model system for studying the bone density processes and biomineralization.

As used herein, the expression of a FAM20C nucleic acid, fragment thereof, can be targeted in a “tissue specific manner” or “cell type specific manner” using a vector in which nucleic acid sequences encoding all or a portion of FAM20C are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific expression of proteins are well known in the art and described herein.

Methods of use for the transgenic mice of the invention are also provided herein. Transgenic mice into which a nucleic acid containing the FAM20C or its encoded protein have been introduced are useful, for example, to develop screening methods to screen therapeutic agents to identify those capable of modulating biomineralization and bone density.

### Pharmaceuticals and Peptide Therapies

The elucidation of the biochemical role played by FAM20C in bone metabolism facilitates the development of pharmaceutical compositions useful for treatment and diagnosis of osteoporosis for example. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

The pharmaceutical preparation is formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

The following materials and methods are provided to facilitate the practice of Example I.

### Molecular Biology

Mouse FAM20C, from 98 bp upstream of the ATG to the last codon, was amplified from cDNA clone BC025826 (Open Biosystems) by PCR, using as primers CAAAGCTTGGACCTTGACCCGCGGGTCGTTG; (SEQ ID NO: 1) and AGCCGCGGCCGCCCCTCTCCGTGGAGGCTCTG (SEQ ID NO: 2), and cloned into HindIII and NotI cut pcDNA3.1/V5-H isB (Invitrogen) to create pcDNA-FAM20C:V5. Mouse FAM20A, from 91 bp upstream of the ATG to the last codon, was amplified from cDNA clone BC029169 (Open Biosystems) by PCR, using as primers GGAATTCTAATCCCCTGTGTGAGCATT (SEQ ID NO: 3) and TTTGGCGGCCGCCGCTCGTCAGATTAGCCTGGC (SEQ ID NO: 4), and cloned into EcoRI and NotI cut pcDNA3.1/V5-H isB to create pcDNA-FAM20A:V5. Mouse FAM20B, from 57 bp upstream of the ATG to the last codon, was amplified from cDNA clone BC019381 (Open Biosystems) by PCR, using as primers CGAATTCTGTTCCCTGTGATAAGCCAG (SEQ ID NO: 5) and GCATGCGGCCGCCCAAGTGGGAGAGTGGCATC (SEQ ID NO: 6), and the resulting fragments was cloned into EcoRI and NotI cut pcDNA3.1/V5-H isB to create pcDNA-FAM20B:V5.

Mouse OPN, from 73 bp upstream of the ATG to the last codon, was amplified from cDNA clone BC057858 (Open Biosystems) by PCR, using as primers AAGGTACCCATCCTTGCTTGGGTTTGCAG (SEQ ID NO: 7) and GTTTTTCCGCGGCCGCCCGTTGACCTCAGAAGATGAAC (SEQ ID NO: 8), and cloned into KpnI and SacII cut pMT(WB)-Ds1-10:FLAG⁸ to create pMT(WB)-OPN:FLAG. pMT(WB)-OPN:FLAG was digested with KpnI and Agel and cloned into KpnI and Agel cut pcDNA3.1/V5-H isB to create pcDNA-OPN:FLAG. Mouse BSP from 66 bp upstream of the ATG to the last codon, was amplified from cDNA clone BC045143 (Open Biosystems) by PCR, using as primers AAGGTACCGAGAACAATCCGTGCCACTC (SEQ ID NO: 9) and GGGAGCGGCCGCCCCTGATGGTAGTAATAATTCTG (SEQ ID NO: 10), and cloned into KpnI and NotI cut pcDNA-OPN:FLAG to create pcDNA-BSP:FLAG. Mouse DMP1, from 39 bp upstream of the ATG to the last codon, was amplified from cDNA clone BC113753 (Open Biosystems) by PCR, using as primers AAGGTACCCCTTGGGAGCCAGAGAGGGTAG (SEQ ID NO: 11) and ACAAGCGGCCGCCCGTAGCCGTCCTGACAGTCATTG (SEQ ID NO: 12), and cloned into KpnI and NotI cut pcDNA-OPN:FLAG to create pcDNA-DMP1:FLAG. Mouse DSPP, from 76 bp upstream of the ATG and to the last codon, was amplified from cDNA clone BC129802 (Open Biosystems) by PCR, using as primers AAGGTACCCCTGGAAAGAGAGATAAGGAAATC (SEQ ID NO: 13) and TTCTGCGGCCGCCCATCATCACTGGTTGAGTGGTTAC (SEQ ID NO: 14), and cloned into KpnI and NotI cut pcDNA-OPN:FLAG to create pcDNA-DSPP:FLAG. Mouse MEPE, from 36 bp upstream of the ATG and to the last codon, was amplified from cDNA clone BC119162 (Open Biosystems) by PCR, using as primers AAGGTACCTCCTGAAGGTGAATGACGCCAG (SEQ ID NO: 15) and AATCGCGGCCGCCCGTCACCATGACTCTCACTAG (SEQ ID NO: 16), and cloned into KpnI and NotI cut pcDNA-OPN:FLAG to create pcDNA-MEPE:FLAG CG31145, from the ATG to the last codon, was amplified from cDNA clone RE73615 bp PCR, using as primers AATGGTACCATGGCCGTCCTGCGTACTATG (SEQ ID NO: 17) and TTATCTAGACGAGGAGACGTCCGTCTCGGATC (SEQ ID NO: 18), and cloned into KpnI and XbaI cut pUASTattB-yki:V5²⁵ and 50 bp upstream of the ATG was added by PCR by using primers CCGCGGCTCGAGGGTACCAAAAAGCCATTTCTGCTGCAAGCAACAACAGTTGCAAC ACCAATCCCATCATGGCCGTCCTG (SEQ ID NO: 19) and CAGGACGGCCATGATGGGATTGGTGTTGCAACTGTTGTTGCTTGCAGCAGAAATGGC TTTTTGGTACCCTCGAGCCGCGG (SEQ ID NO: 20) to create UASattB-CG31145:V5. CG3631, from the ATG to the last codon, was amplified from cDNA isolated from S2 cells by PCR, using as primers GGCGGTACCATGAACAAGCGCAGCGTCATCATC (SEQ ID NO: 21) and CTGTCTAGACAGAGTTTTGAACATTTTGTC (SEQ ID NO: 22), and cloned into KpnI and XbaI cut pUASTattB-yki:V5 and 50 bp upstream of the ATG was added by PCR by using primers GGCTCGAGGGTACCCTGCGTATACGTAATATTAAAAATAGGCTAACGCCCGCCCAG GCTGCAGGATGAACAAGCGCAG (SEQ ID NO: 23) and CTGCGCTTGTTCATCCTGCAGCCTGGGCGGGCGTTAGCCTATTTTTAATATTACGTAT ACGCAGGGTACCCTCGAGCC (SEQ ID NO: 24) to create UASattB-CG3631:V5.

Site-specific mutagenesis was performed by PCR essentially as described²⁶, by using primers CATCCACTTGGGCGGCGGGCGCGGG and CCCGCGCCCGCCGCCCAAGTGGATG (SEQ ID NO: 25) (FAM20C^{GG}), TGGGGAACATGGGTCGGCATCACTAC (SEQ ID NO: 26) and GTAGTGATGCCGACCCATGTTCCCCA (FAM20C^{D453G}) (SEQ ID NO: 27), GCATGCCCTGTGTAGGAGGCCCGAC (SEQ ID NO: 28) and GTCGGGCCTCCTACACAGGGCATGC (FAM20C^{G374R}) (SEQ ID NO: 29), CATGCCCTGTGTGAGAGGCCCGACCA and TGGTCGGGCCTCTCACACAGGGCATG (FAM20C^{G374E}) (SEQ ID NO: 30), ATCGAAGGATCCCGGGCGGCCTTCC (SEQ ID NO: 31) and GGAAGGCCGCCCGGGATCCTTCGAT (FAM20C^{E383R}) (SEQ ID NO: 32), GCCCTGGACCGGTGGTTGCGCATAG (SEQ ID NO: 33) and CTATGCGCAACCACCGGTCCAGGGC (FAM20C^{R544W}) (SEQ ID NO: 34), CACAACCCAGCCAACGATGCCTTACTG (SEQ ID NO: 35) and CAGTAAGGCATCGTTGGCTGGGTTGTG (FAM20C^{1241N})(SEQ ID NO: 36), CCATGAAGTCAAGGGGCACGCAGCTG (SEQ ID NO: 37) and CAGCTGCGTGCCCCTTGACTTCATGG (FAM20C^{G261R}) (SEQ ID NO: 38), GATATGACCGTCTTTAATTTCCTCATGG (SEQ ID NO: 39) and CCATGAGGAAATTAAAGACGGTCATATC (FAM20C^{D446N}) (SEQ ID NO:40). All mutations were confirmed by DNA sequencing.

### Cell Culture and Biochemistry

Human embryonic kidney (HEK293T) cells were grown in DMEM/F12 medium (Invitrogen) containing 10% fetal bovine serum. Lipofectamine (Invitrogen) was used for transfection. 1 μg of DNA was used for 1 well of 6 well plate. After 2 to 4 days induction, conditioned media (5 ml for 1 well of 6 well plate) was collected and centrifuged (3000 rpm, 15 min).

For secretion assays, condition media and cell lysates were subjected to Western blotting to detect FAM20C:V5, FAM20A:V5, and FAM20B:V5. GFP:V5 was used as a non-secreted control protein.

For mobility shift assays on OPN:FLAG, BSP:FLAG, DMP1:FLAG, DSPP:FLAG and MEPE:FLAG, conditioned media were subjected SDS-PAGE, and OPN:FLAG, BSP:FLAG, DMP1:FLAG, DSPP:FLAG and MEPE:FLAG were detected by Western blotting.

For FAM20C:V5 purification, pcDNA-FAM20C:V5 was transfected to HEK293T cells, and FAM20C:V5 was purified from conditioned media using anti-V5 agarose beads (Sigma). Condition media were incubated with anti-V5 affinity gel (Sigma) overnight at 4° C. Agarose beads were collected by centrifugation, and washed 5 times with TBS. Proteins were eluted using V5 peptides in TBS (Sigma, final concentration: 100 μg/ml). For purification of OPN:FLAG, BSP:FLAG, DMP1:FLAG, DSPP:FLAG and MEPE:FLAG, condition media were incubated with anti-FLAG M2 affinity gel (Sigma) overnight at 4° C. Agarose beads were collected by centrifugation, and washed 5 times with TBS. Purified quantification was performed on gels stained by SYPRO Ruby Protein Gel Stain (Invitrogen), using BSA as a standard.

S2 cells were grown in Schneider's Drosophila medium (Invitrogen) containing 10% fetal bovine serum. Cellufectin (Invitrogen) was used for transfection. 1 μg of UASattB-CG31145:V5 or UASattB-CG3631:V5 and 0.5 μg of pWAGa14 (a kind gift from Y. Hiromi) were co-transfected for 1 well of 6 well plate. After 2 days induction, conditioned media (5 ml for 1 well of 6 well plate) and cell lysate were collected.

For Western blotting, HRP-conjugated mouse anti-V5 (1:10,000, Invitrogen), and HRP-conjugated mouse anti-FLAG M2 (1:10,000, Sigma) were used. Proteins were transferred to nitrocellulose membranes (Bio-Rad Labolatories) and detected using Super Signal West Dura (Pierce).

### Kinase Assays

FAM20C kinase assays were performed in 10 μl reactions, with 500 μM ATP, including 3 μCi (0.05 μM) of [gamma-³²P]ATP (6000 Ci/mmol, PerkinElmer), 50 mM Tris (pH 7.0), 10 mM MnCl₂, 0.1% BSA, purified FAM20C:V5 (5 ng, 0.066 μmol), and dephosphorylated alpha-Casein (0.1 μg, Sigma). The kinase reaction was linear at 5 minutes, and the reaction rate was dependent on enzyme and substrate concentrations. Reactions were stopped by adding SDS sample buffer and boiling, and one half of the reaction volume was subjected to SDS-PAGE. Radioactivity within the gels was detected using a Molecular Dynamics Phosphor Imager. For quantitation of gamma-³²P incorporation, bands were cut out of the gels using the Phosphor Imager pictures as a reference, and then counted by liquid scintillation using a Beckman Coulter LC6500. Myelin Basic Protein (0.5 μg, NEB) and affinity-purified Fat2-3:FLAG (0.1 μg)⁸ were used as control substrates. To determine pH-dependency, the pH in the reaction buffer was varied using Tris-HCl buffers. Synthesized peptides were designed from the sequence of human Osteopontin 115Asp to 132Leu, adding three basic amino acids (Arg-Lys-Arg) to its N-terminus to enable peptide binding to phosphocellulose paper (P81, Upstate). Peptides were purchased from Peptide2.0. Peptide1 (RKRDDSHQSDESHHSDESDEL; SEQ ID NO: 41), Peptide2 (RKRDDAHQADEAHHADEADEL; SEQ ID NO: 42), Peptide3 (RKRDDSHQADESHHADEADEL; SEQ ID NO: 43), and Peptide4 (RKRDDAHQSDEAHHSDESDEL; SEQ ID NO: 44).

FAM20C kinase assay with peptides substrates were performed in 10 μl reactions, with 500 μM ATP, including 3 μCi (0.05 μM) of [gamma-³²P]ATP (6000 Ci/mmol, PerkinElmer), 50 mM Tris (pH 7.0), 10 mM MnCl₂, 0.1% BSA, purified FAM20C:V5 and peptides. Casein Kinase I (500 units, NEB) was used as a control kinase. mutant versions (5 ng, 0.066 μmol), and peptides (2.5 μg, 1 nmol as a standard condition. 250 ng, 100 μmol in FAM20C mutants). Reactions were stopped by adding 1.5 μl 0.1N—HCl, and one half of the reaction volume was pipetted on to P81 phospho-cellulose filter paper²⁷. The filter papers were washed with 0.5% phosphoric acid and subjected to liquid scintillation counting.

### Immunostaining

HEK293T cells were plated on a tissue culture slide (8 chamber, Thermo Fisher Scientific), and pcDNA-FAM20C:V5 or its mutant forms was transfected. For S2 cells, UASattB-CG31145:V5 or UASattB-CG3631:V5 and pWAGa14 were co-transfected in the tissue culture slide. 48 hours after transfection, the cells were fixed with 4% of paraformaldehyde in PBS, and immuno-stained. Mouse anti-V5 (1:500, Invitrogen), Rabbit anti-V5 (1:500, Bethyl Laboratories), Rabbit anti-Giantin (1:500, Abcam), Mouse anti-KDEL (1:250, Stressgen), mouse anti-Drosophila Golgi (7H6D7C2, 1:500, EMD Biosciences), and rabbit anti-Drosophila GM130 (1:200, abcam) were used as primary antibodies. Alexa Fluor 488-conjugated goat anti-mouse or anti-rabbit IgGs (1:100, Invitrogen), and Cy3-conjugated anti-mouse or anti-rabbit IgGs (1:100, Jackson Immuno Research Laboratories) were used as secondary antibodies. Images were obtained on a confocal microscope (FV-1000D, Olympus).

The following examples are provided to illustrate certain embodiments of the invention. They are not intended to limit the invention in any way.

### Example I

### Biochemical Characterization of FAM20C

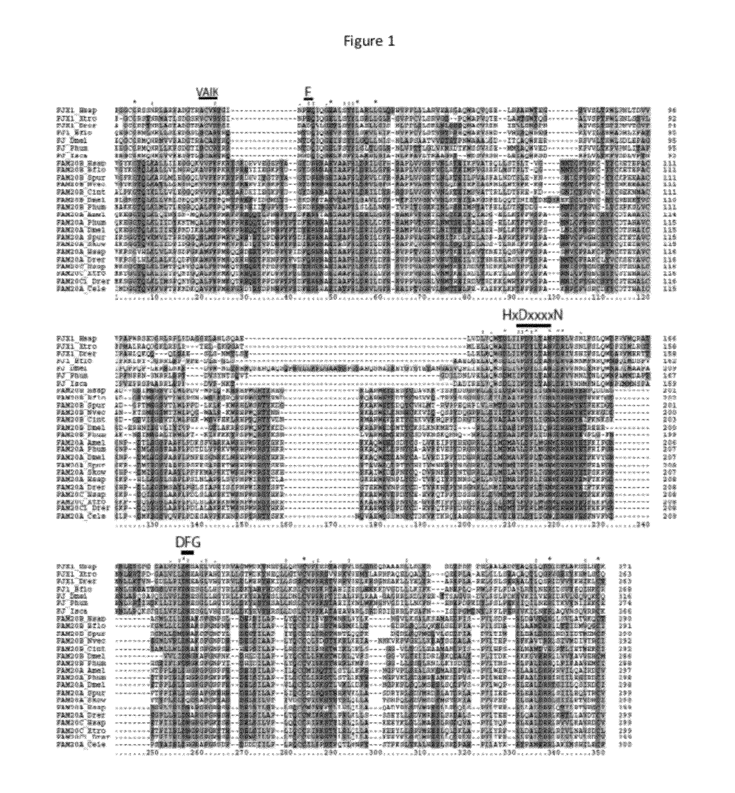

In earlier research, we identified Drosophila Four jointed (Fj) as a Golgi-localized protein kinase that phosphorylates cadherin domains of the transmembrane receptor and ligand of the Drosophila Fat signaling pathway, Fat and Dachsous⁸. As Fj exhibits only very limited sequence similarity to known protein kinases, and was the first molecularly identified Golgi-localized protein kinase, it defined a new class of protein kinases. To identify other potential Golgi kinases, we conducted bioinformatic searches for genes encoding proteins related to Fj and its mammalian homologue, Fjx1. The closest homologues in humans are encoded by Family with sequence similarity 20 (FAM20), which comprises FAM20A, FAM20B, and FAM20C⁹. Two members of this protein family were also identified in Drosophila, encoded by CG31145 and CG3631 (FIG. 1a). Sequence analysis identifies them as potential type II transmembrane proteins, which is often a feature of Golgi-resident proteins. Amino acid sequence comparisons also revealed the presence of conserved sequence motifs typical of kinases, including amino acids implicated in catalysis and in binding metal ion co-factors (FIG. 1). These observations suggested that these proteins could encode novel kinases of the secretory pathway. Consistent with this, FAM20B was recently identified as a kinase that phosphorylates xylose within the glycosaminoglycan core linker¹⁰

To facilitate biochemical characterization of FAM20 proteins, we constructed V5-epitope tagged versions of murine FAM20 genes and expressed them in cultured mammalian cells. Earlier studies of FAM20C (also known as DMP4) identified it as a secreted protein^{7,9}.

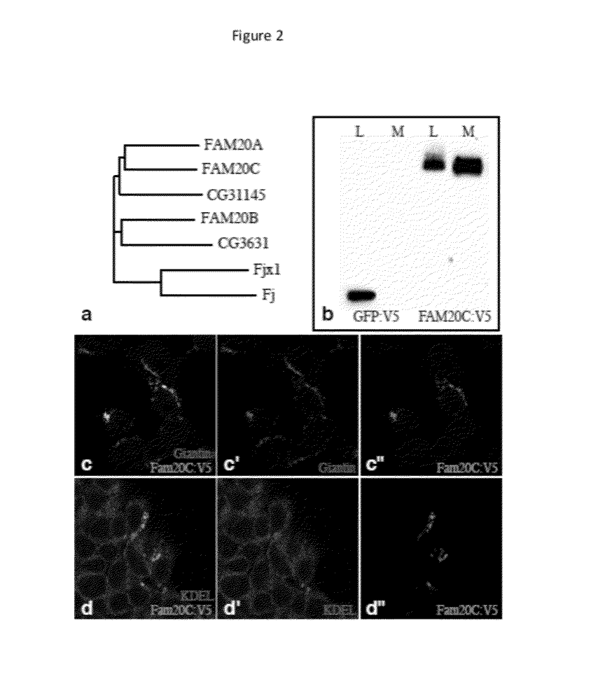

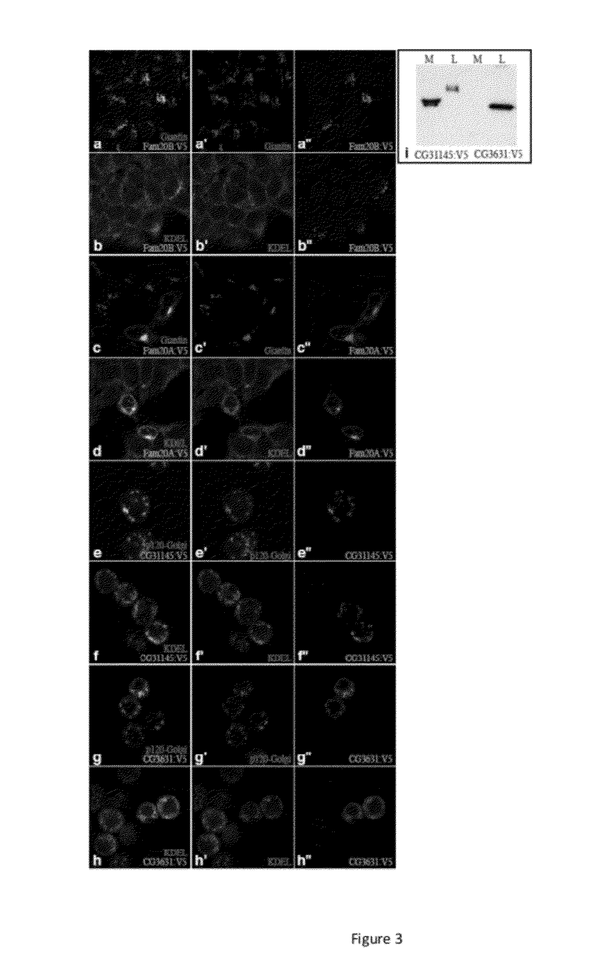

However, some Golgi-resident transmembrane proteins, including glycosyltransferases¹¹, and the kinase Fj¹², are also secreted from cells, typically by cleavage of a proteolytically sensitive stem between the transmembrane domain and the catalytic domain. Western blotting on cell lysates and conditioned media of transfected cells revealed that FAM20C was present both in the medium and cell lysate (FIG. 2b), whereas FAM20A and FAM20B were only detected in the cell lysate (Not shown). Immunolocalization revealed that FAM20C and FAM20B exhibited substantial overlap with a Golgi marker, whereas FAM20A exhibited substantial overlap with an ER marker (FIGS. 2c, 3). In Drosophila cells, CG31145 overlapped a Golgi marker and was secreted from cells, whereas CG3631 largely overlapped an ER marker and was not secreted (FIG. 3).

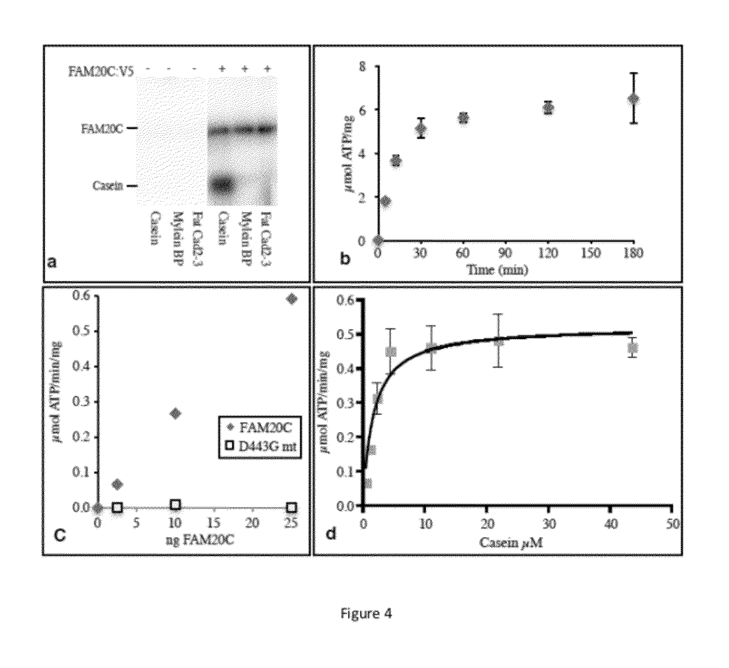

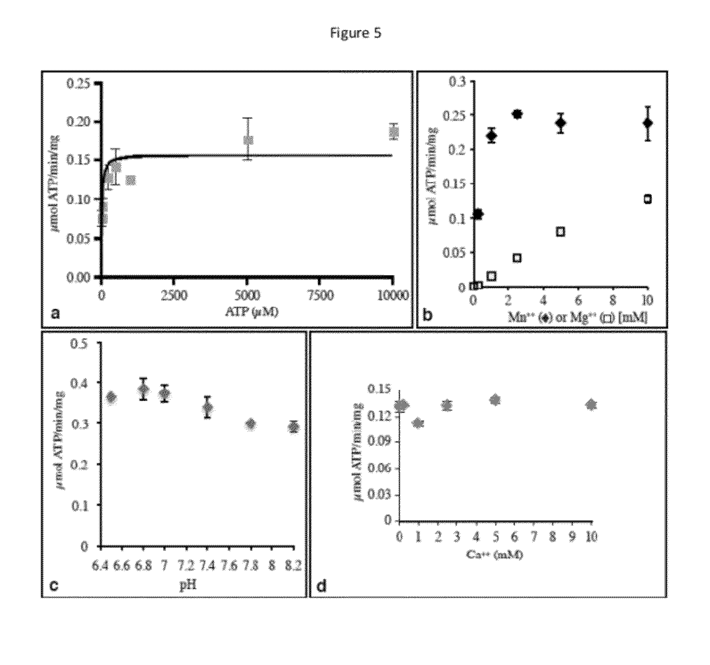

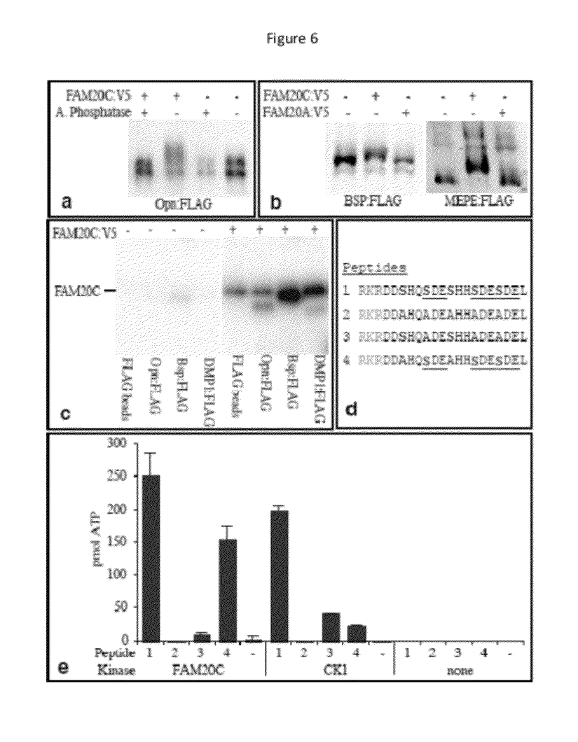

To examine FAM20C for potential protein kinase activity, we first assayed FAM20C:V5 secreted into the medium. Many protein kinases, including Fj, can undergo an autophosphorylation reaction. Thus, we incubated condition media from FAM20C:V5-expressing HEK293T cells with radiolabeled ATP. This revealed that FAM20C:V5 was phosphorylated (Not shown), consistent with the possibility that it encodes a kinase. To further characterize this apparent kinase activity, we affinity purifed FAM20C:V5 from conditioned medium through the V5-epitope tag, and used it as an enzyme source for in vitro kinase assays. As one candidate FAM20C substrate, we used a dephosphorylated form of alpha-Casein, an abundant secreted phosphoprotein in milk. Dephosphorylated Casein was phosphorylated by purified FAM20C in vitro, as was FAM20C itself, establishing FAM20C as a Casein kinase (FIG. 4a). CG31145 also has Casein kinase activity (Not shown). Conversely, neither Myelin basic protein, a common substrate for kinases that recognize basic sequence motifs, and nor cadherin domains of Fat that are substrates for Fj, were detectably phosphorylated by FAM20C (FIG. 4a). Using Casein as a model substrate, we characterized parameters of FAM20C kinase activity. Mutation of a highly conserved Asp near the catalytic center abolished kinase activity, indicating that kinase activity is associated with FAM20C rather than a co-purifying contaminant (FIG. 4c). Under linear reaction conditions (5 minute assays, FIG. 4b), and averaging the results from several independent experiments, we obtained a Michaelis constant (Km) of FAM20C for Casein of 1.5 μM, a Km for ATP of 78 μM, a turnover number (kcat) of 52 per minute, and a V. of 0.7 μM ATP/min/mg FAM20C (FIGS. 4, 5 and data not shown). These observations confirm that FAM20C acts catalytically. Although most kinases have higher activity with Mg²⁺ as a cofactor, FAM20C exhibits a preference for Mn²⁺ (FIG. 5), similar to the Golgi kinase Fj⁸. FAM20C is active over a wide pH range, but has slightly higher activity at or below pH 7.0 (FIG. 5). FAM20C has been been identified as a Ca²⁺-binding protein⁷, but the presence or absence of Ca²⁺ did not significantly affect its kinase activity (FIG. 5). The Small Integrin-Binding Ligand N-linked Glycoproteins (SIBLINGs) are a family of five related proteins, osteopontin (OPN), bone sialoprotein (BSP), dentin matrix protein 1 (DMP1), dentin sialophosphoprotein (DSPP) and matrix extracellular phosphoglycoprotein (MEPE), each of which contain an integrin binding motif and are secreted phosphoproteins¹³⁻¹⁵. They are highly expressed in bone and teeth, and have been implicated in modulating biomineralization through both genetic and biochemical studies. Moreover, their ability to modulate biomineralization can be affected by their phosphorylation status^{13,16-19}. To investigate whether SIBLING proteins are substrates of FAM20C kinase activity, we co-expressed FLAG epitope-tagged OPN, BSP or MEPE together with FAM20C:V5 in cultured HEK293T cells. Co-expression of FAM20C, but not FAM20A, decreased their mobility on SDS-PAGE gels (FIG. 6), consistent with the hypothesis that their phosphorylation is increased. Incubation of FAM20C-modified OPN:FLAG with phosphatase reversed this FAM20C-dependent mobility shift (FIG. 6). Thus, phosphorylation of SIBLING proteins can be promoted by FAM20C in vivo. To confirm that this involves a direct phopshorylation of SIBLING proteins by FAM20C, we performed in vitro kinase assays on three different affinity purified SIBLING proteins. Transfer of ³²P onto OPN, BSP, and DMP1 was observed in the presence of FAM20C:V5, indicating that FAM20C can directly phosphorylate SIBLING proteins (FIG. 6).

Two families of ubiquitously expressed protein kinases have been termed Casein kinase I (CKI) and Casein Kinase II (CKII). However, their contribution to biological phosphorylation of Caseins is uncertain, as Caseins are secreted proteins, whereas CKI and CKII proteins are predominantly cytoplasmic and nuclear. A distinct enzymatic activity that could be responsible for endogenous casein phosphorylation, termed Golgi apparatus casein kinase (G-CK) was first identified in lactating mammary glands²⁰. CKI, CKII, and G-CK share a preference for acidic sequence motifs as concensus phosphorylation sites, but differ in their site preferences²¹. The majority of phosphorylation sites identified in OPN conform to a concensus sequence for G-CK (S/T-X-D/E/^{P}S)²², and G-CK can phosphorylate OPN²³. Intriguingly, the Km for ATP of FAM20C (78 μM) is similar to that originally measured for G-CK (80 μM)²⁰. The Km for α-Casein is higher (12 μM for G-CK)²⁰, but this could reflect differences in assay conditions, or in the quality or purity of this substrate. G-CK also exhibits a preference for Mn⁺⁺ over Mg⁺⁺ as a cofactor²⁴.

To investigate the site specificity of FAM20C, we assayed phosphorylation of a peptide including amino acids 115-132 of human OPN(OPN-ASARM, for acidic, serine- and asparatate-rich motif)¹⁶. In its phosphorylated form, the OPN-ASARM peptide functions as an inhibitor of mineralization through binding to hydroxyapaptite¹⁶. It contains 5 serine residues, three of which are important for inhibiting biomineralization¹⁶. Intriguingly, these three Ser residues all conform to the consensus sequence for G-CK (FIG. 6). We used this peptide, and derivatives with Ser to Ala mutations, to investigate the site specificity of FAM20C on a functionally important substrate. In vitro kinase reactions confirmed that OPN-ASARM is a FAM20C substrate, whereas a derivative with all five Ser residues replaced by Ala (OPN-ASARM^{5S-A}) was not phosphorylated (FIG. 6). A peptide in which only the three G-CK concensus sites were changed to Ala (OPN-ASARM^{3S-A}) was phosphorylated much less effectively, whereas a derivative in which the other two Ser residues were changed to Ala (OPN-ASARM^{2S-A}) was still efficiently phosphorylated by FAM20C. We also examined phosphorylation of these peptides by CKI. CKI also phosphorylated OPN-ASARM, and had reduced, but similar levels of activity on both OPN-ASARM^{3S-A} and OPN-ASARM^{2S-A}. Together these observations imply that FAM20C acts preferentially at G-GK consensus sites. Together with its normal Golgi localization, and other enzymatic properties, these observations identify it as a G-CK. Although G-CK was first described as an enzymatic activity almost 40 years ago²⁰, its molecular identity has remained elusive, and FAM20C is the first G-CK to be molecularly identified. As we were unable to detect G-CK activity in association with either FAM20A or FAM20B, it's possible that FAM20C is the sole mammalian G-CK protein.

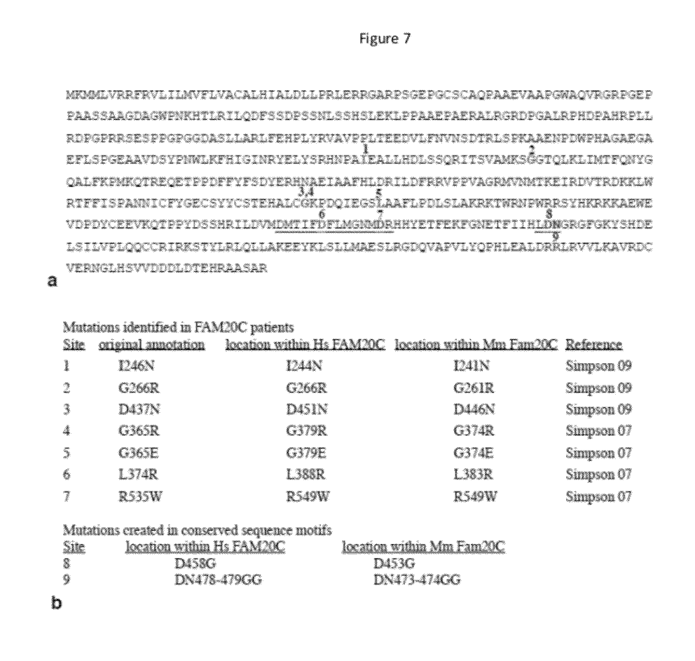

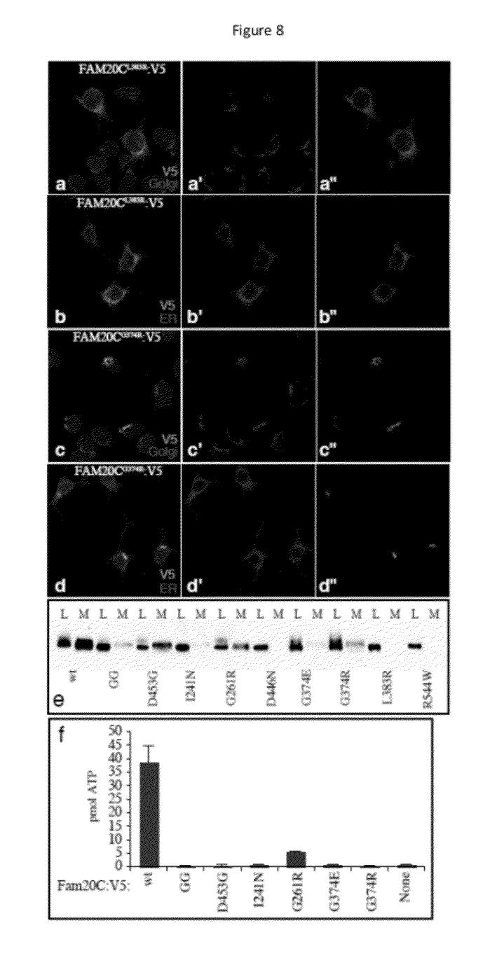

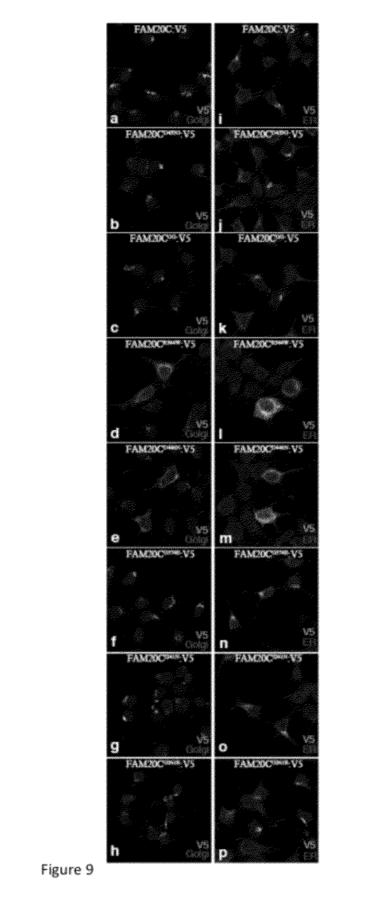

If FAM20C modulates bone formation by acting as a protein kinase, then mutations identified in human Raine syndrome patients should impair FAM20C kinase activity. To examine this, we engineered such mutations (corresponding to human FAM20C G374R, G374E, L383R, R544W, 1241N, G261R, D446N) into recombinant mouse FAM20C:V5 expressed in cultured cells (FIG. 7). For comparison we also examined FAM20C with a mutation in a conserved Asp residue described above (D453G), and FAM20C with a mutation in a motif predicted to bind metal ion cofactors (DN473-474GG). These mutations in conserved motifs abolished catalytic activity with disrupting FAM20C localization. Three of the mutations identified in patients (L383R, R544W, and D446N) resulted in mislocalization of protein from the Golgi to the ER, and an absence of detectable protein secreted into the media (FIGS. 8, 9). Since misfolded secretory pathway proteins are typically retained in the ER, these observations suggest that these mutations result in misfolded protein, and we did not attempt to purify them.

The remaining four mutant isoforms exhibited Golgi localization and were secreted into the medium, albeit less efficiently than wild-type FAM20C (FIG. 8). Three of these (G374E, G374R, and 1241N) had no detectable kinase activity, whilst the fourth (G261R) had diminished kinase activity. Thus, all four point mutations that did not appear to be associated with gross mis-folding of FAM20C protein impaired kinase activity, supporting the importance of kinase activity to FAM20C function in vivo. G261R mutation in humans is associated with a non-lethal form of Raine syndrome, whereas homozygosity for G374 mutations was associated with the more severe, lethal form of this disease, which suggests that the extent of impairment of kinase activity and could correlate with disease severity.

Our observations identify FAM20C as a novel, Golgi-localized protein kinase that plays a crucial role in modulating bone formation. We also identify SIBLING proteins as an important class of FAM20C substrates. Since SIBLING proteins can act as inhibitors of biomineralization depending upon their phosphorylation state^{13,16-19}, the increased bone density of Raine syndrome patients could be accounted for by decreased phosphorylation of SIBLING proteins. Kinase inhibitors have emerged in recent years as important and effective therapeutics for human diseases. Since mutations that impair FAM20C kinase activity increase bone formation, inhibitors of FAM20C kinase activity would be expected to have similar effects. Thus, such inhibitors might be used to treat diseases associated with reduced bone density, such as osteoporosis. As OPN and other SIBLING proteins have also been linked to multiple stages of cancer progression, including metastasis¹⁵, modulation of FAM20C kinase activity also has potential implications for cancer therapy.

### REFERENCES

1. Hulskamp, G., et al. Raine syndrome: report of a family with three affected sibs and further delineation of the syndrome. Clin Dysmorphol 12, 153-160 (2003).
2. Raine, J., Winter, R. M., Davey, A. & Tucker, S. M. Unknown syndrome: microcephaly, hypoplastic nose, exophthalmos, gum hyperplasia, cleft palate, low set ears, and osteosclerosis. J Med Genet. 26, 786-788 (1989).
3. Rejjal, A. Raine syndrome. Am J Med Genet. 78, 382-385 (1998).
4. Fradin, M., et al. Osteosclerotic bone dysplasia in siblings with a Fam20C mutation. Clin Genet (2010).
5. Simpson, M. A., et al. Mutations in FAM20C are associated with lethal osteosclerotic bone dysplasia (Raine syndrome), highlighting a crucial molecule in bone development. American journal of human genetics 81, 906-912 (2007).
6. Simpson, M. A., et al. Mutations in FAM20C also identified in non-lethal osteosclerotic bone dysplasia. Clin Genet. 75, 271-276 (2009).
7. Hao, J., Narayanan, K., Muni, T., Ramachandran, A. & George, A. Dentin matrix protein 4, a novel secretory calcium-binding protein that modulates odontoblast differentiation. The Journal of biological chemistry 282, 15357-15365 (2007).
8. Ishikawa, H. O., Takeuchi, H., Haltiwanger, R. S. & Irvine, K. D. Four-jointed is a Golgi kinase that phosphorylates a subset of cadherin domains. Science (New York, N.Y. 321, 401-404 (2008).
9. Nalbant, D., et al. FAM20: an evolutionarily conserved family of secreted proteins expressed in hematopoietic cells. BMC genomics 6, 11 (2005).
10. Koike, T., Izumikawa, T., Tamura, J. & Kitagawa, H. FAM20B is a kinase that phosphorylates xylose in the glycosaminoglycan-protein linkage region. The Biochemical journal 421, 157-162 (2009).
11. El-Battari, A., et al. Different glycosyltransferases are differentially processed for secretion, dimerization, and autoglycosylation. Glycobiology 13, 941-953 (2003).
12. Buckles, G. R., Rauskolb, C., Villano, J. L. & Katz, F. N. four-jointed interacts with dachs, abelson and enabled and feeds back onto the Notch pathway to affect growth and segmentation in the Drosophila leg. Development 128, 3533-3542. (2001).
13. Qin, C., Baba, O. & Butler, W. T. Post-translational modifications of sibling proteins and their roles in osteogenesis and dentinogenesis. Crit. Rev Oral Biol Med 15, 126-136 (2004).
14. Fisher, L. W. & Fedarko, N. S. Six genes expressed in bones and teeth encode the current members of the SIBLING family of proteins. Connective tissue research 44 Suppl 1, 33-40 (2003).
15. Bellahcene, A., Castronovo, V., Ogbureke, K. U., Fisher, L. W. & Fedarko, N. S. Small integrin-binding ligand N-linked glycoproteins (SIBLINGs): multifunctional proteins in cancer. Nature reviews 8, 212-226 (2008).
16. Addison, W.N., Masica, D. L., Gray, J. J. & McKee, M. D. Phosphorylation-dependent inhibition of mineralization by osteopontin ASARM peptides is regulated by PHEX cleavage. J Bone Miner Res 25, 695-705 (2010).
17. Gericke, A., et al. Importance of phosphorylation for osteopontin regulation of biomineralization. Calcified tissue international 77, 45-54 (2005).
18. Jono, S., Peinado, C. & Giachelli, C. M. Phosphorylation of osteopontin is required for inhibition of vascular smooth muscle cell calcification. The Journal of biological chemistry 275, 20197-20203 (2000).
19. Kazanecki, C. C., Uzwiak, D. J. & Denhardt, D. T. Control of osteopontin signaling and function by post-translational phosphorylation and protein folding. Journal of cellular biochemistry 102, 912-924 (2007).
20. Bingham, E. W. & Farrel, H. M., Jr. Casein kinase from the Golgi apparatus of lactating mammary gland. The Journal of biological chemistry 249, 3647-3651 (1974).
21. Lasa-Benito, M., Marin, O., Meggio, F. & Pinna, L. A. Golgi apparatus mammary gland casein kinase: monitoring by a specific peptide substrate and definition of specificity determinants. FEBS letters 382, 149-152 (1996).
22. Sorensen, E. S., Hojrup, P. & Petersen, T. E. Posttranslational modifications of bovine osteopontin: identification of twenty-eight phosphorylation and three O-glycosylation sites. Protein Sci 4, 2040-2049 (1995).
23. Lasa, M., Chang, P. L., Prince, C. W. & Pinna, L. A. Phosphorylation of osteopontin by Golgi apparatus casein kinase. Biochemical and biophysical research communications 240, 602-605 (1997).
24. Bingham, E. W. & Groves, M. L. Properties of casein kinase from lactating bovine mammary gland. The Journal of biological chemistry 254, 4510-4515 (1979).
25. Oh, H. & Irvine, K. D. In vivo analysis of Yorkie phosphorylation sites. Oncogene 28, 1916-1927 (2009).
26. Hemsley, A., Arnheim, N., Toney, M. D., Cortopassi, G. & Galas, D. J. A simple method for site-directed mutagenesis using the polymerase chain reaction. Nucleic Acids Res 17, 6545-6551 (1989).
27. Hardie, D. G. Peptide assay of protein kinases and use of variant peptides to determine recognition motifs. Methods Mol Biol 99, 191-201 (2000).

### Example II

### Screening Assays for the Identification of Agents which Modulate FAM20C Action for Therapeutic Benefit

Certain aspects of the present disclosure provide methods of screening for a candidate drug (agent or compound) that modulates FAM20C interactions and associated pathology. Various types of candidate drugs may be screened by the methods described herein, including nucleic acids, polypeptides, small molecule compounds, and peptidomimetics. In a preferred approach, putative therapeutic molecules can be screened in vitro using suitable substrates and purified FAM20C protein as exemplified in Example I. Preferably, in vitro screening can be performed in high throughput format. In some cases, genetic agents can be screened by contacting the cell with a nucleic acid construct coding for a gene. For example, one may screen cDNA libraries expressing a variety of genes, to identify other genes that modulate FAM20C-SIBLING phosphorylation reactions and subsequent signal transduction. For example, the identified drugs may modulate FAM20C SIBLING binding reactions, subcellular localization and/or cell morphology, density or viability. Accordingly, irrespective of the exact mechanism of action, drugs identified by the screening methods described herein are expected to provide therapeutic benefit to patients suffering from a variety of bone disorders.

Screening methods described herein use may employ in vitro kinase assays or a variety of cell types. Candidate drugs can be screened from large libraries of synthetic or natural compounds. One example is an FDA approved library of compounds that can be used by humans. In addition, compound libraries are commercially available from a number of companies including but not limited to Maybridge Chemical Co. (Trevillet, Cornwall, UK), Comgenex (Princeton, N.J.), Microsource (New Milford, Conn.), Aldrich (Milwaukee, Wis.), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, Calif.), ChemDiv, (San Diego, Calif.), Chemical Block Lt, (Moscow, Russia), ChemStar (Moscow, Russia), Exclusive Chemistry, Ltd (Obninsk, Russia), Enamine (Kiev, Ukraine), Evotec (Hamburg, Germany), Indofine (Hillsborough, N.J.), Interbioscreen (Moscow, Russia), Interchim (Montlucon, France), Life Chemicals, Inc. (Orange, Conn.), Microchemistry Ltd. (Moscow, Russia), Otava, (Toronto, ON), PharmEx Ltd. (Moscow, Russia), Princeton Biomolecular (Monmouth Junction, N.J.), Scientific Exchange (Center Ossipee, N.H.), Specs (Delft, Netherlands), TimTec (Newark, Del.), Toronto Research Corp. (North York ON), UkrOrgSynthesis (Kiev, Ukraine), Vitas-M, (Moscow, Russia), Zelinsky Institute, (Moscow, Russia), and Bicoll (Shanghai, China). Combinatorial libraries are available and can be prepared. Libraries of natural compounds in the form of bacterial, fungal, plant and animal extracts are commercially available or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

For example, the cells in Example 1 can be incubated in the presence and absence of a test compound and the effect of the compound on FAM20C phosphorylation activity on relevant substrates assessed. Agents so identified could then be tested in whole animal models to assess in vivo efficacy.

Agents identified using the screening assays described herein are also encompassed by the present invention While the invention has been described in detail and with reference to specific examples thereof, it will be apparent to one skilled in the art that various changes and modifications can be made therein without departing from the spirit and scope thereof.

What is claimed is:

1. A method for identifying an agent that modulates FAM20C protein kinase activity, comprising:
a) incubating FAM20C protein and at least one substrate under conditions effective for kinase action to occur, in the presence and absence of said agent; and
b) assessing phosphorylation level on said substrate, agents which alter the phosphorylation level of said substrate relative to FAM20C kinase incubated in the absence of said agent being effective to modulate FAM20C kinase activity.

2. The method of claim 1 wherein said agent inhibits FAM20C kinase activity.

3. The method of claim 1, wherein said agent augments FAM20C kinase activity.

4. The method of claim 1, wherein said substrate is a SIBLING protein or a phosphorylatable fragment thereof.

5. The method of claim 4, wherein said SIBLING protein is selected from the group consisting of osteopontin (OPN), bone sialoprotein (BSP), dentin matrix protein 1 (DMP1), dentin sialophosphoprotein (DSPP) and matrix extracellular phosphoglycoprotein (MEPE).

6. The method of claim 1, wherein the agent is selected from the group consisting of a naturally occurring or synthetic polypeptide or oligopeptide, a peptidomimetic, a small organic molecule, a polysaccharide, a lipid, a fatty acid, a polynucleotide, an RNAi or siRNA, an asRNA, and an oligonucleotide.

7. The method of claim 1 wherein the contacting is in vitro.

8. The method of claim 1 wherein the contacting is in vivo.

9. The method of claim 8, wherein the effect of said agent on bone density or bone mineralization is assessed.

10. The method of claim 8, further comprising determining whether said agent alters the intracellular localization of FAM20C.

11. An agent identified by the method of claim 1.


SEQUENCE LISTING (72 sequences)

SEQ ID NO: 1 - DNA, 31 bp, Artificial Sequence
  Primer
caaagcttggaccttgacccgcgggtcgttg

SEQ ID NO: 2 - DNA, 32 bp, Artificial Sequence
  Primer
agccgcggccgcccctctccgtggaggctctg

SEQ ID NO: 3 - DNA, 27 bp, Artificial Sequence
  Primer
ggaattctaatcccctgtgtgagcatt

SEQ ID NO: 4 - DNA, 33 bp, Artificial Sequence
  Primer
tttggcggccgccgctcgtcagattagcctggc

SEQ ID NO: 5 - DNA, 27 bp, Artificial Sequence
  Primer
cgaattctgttccctgtgataagccag

SEQ ID NO: 6 - DNA, 32 bp, Artificial Sequence
  Primer
gcatgcggccgcccaagtgggagagtggcatc

SEQ ID NO: 7 - DNA, 29 bp, Artificial Sequence
  Primer
aaggtacccatccttgcttgggtttgcag

SEQ ID NO: 8 - DNA, 38 bp, Artificial Sequence
  Primer
gtttttccgcggccgcccgttgacctcagaagatgaac

SEQ ID NO: 9 - DNA, 28 bp, Artificial Sequence
  Primer
aaggtaccgagaacaatccgtgccactc

SEQ ID NO: 10 - DNA, 35 bp, Artificial Sequence
  Primer
gggagcggccgcccctgatggtagtaataattctg

SEQ ID NO: 11 - DNA, 30 bp, Artificial Sequence
  Primer
aaggtaccccttgggagccagagagggtag

SEQ ID NO: 12 - DNA, 36 bp, Artificial Sequence
  Primer
acaagcggccgcccgtagccgtcctgacagtcattg

SEQ ID NO: 13 - DNA, 32 bp, Artificial Sequence
  Primer
aaggtacccctggaaagagagataaggaaatc

SEQ ID NO: 14 - DNA, 37 bp, Artifical Sequence
  Primer
ttctgcggccgcccatcatcactggttgagtggttac

SEQ ID NO: 15 - DNA, 30 bp, Artificial Sequence
  Primer
aaggtacctcctgaaggtgaatgacgccag

SEQ ID NO: 16 - DNA, 34 bp, Artificial Sequence
  Primer
aatcgcggccgcccgtcaccatgactctcactag

SEQ ID NO: 17 - DNA, 30 bp, Artificial Sequence
  Primer
aatggtaccatggccgtcctgcgtactatg

SEQ ID NO: 18 - DNA, 32 bp, Artificial Sequence
  Primer
ttatctagacgaggagacgtccgtctcggatc

SEQ ID NO: 19 - DNA, 80 bp, Artificial Sequence
  Primer
ccgcggctcgagggtaccaaaaagccatttctgctgcaagcaacaacagttgcaacaccaatcccatcatggccgtcctg

SEQ ID NO: 20 - DNA, 80 bp, Artificial Sequence
  Primer
caggacggccatgatgggattggtgttgcaactgttgttgcttgcagcagaaatggctttttggtaccctcgagccgcgg

SEQ ID NO: 21 - DNA, 33 bp, Artificial Sequence
  Primer
ggcggtaccatgaacaagcgcagcgtcatcatc

SEQ ID NO: 22 - DNA, 30 bp, Artificial Sequence
  Primer
ctgtctagacagagttttgaacattttgtc

SEQ ID NO: 23 - DNA, 78 bp, Artificial Sequence
  Primer
ggctcgagggtaccctgcgtatacgtaatattaaaaataggctaacgcccgcccaggctgcaggatgaacaagcgcag

SEQ ID NO: 24 - DNA, 78 bp, Artificial Sequence
  Primer
ctgcgcttgttcatcctgcagcctgggcgggcgttagcctatttttaatattacgtatacgcagggtaccctcgagcc

SEQ ID NO: 25 - DNA, 25 bp, Artificial Sequence
  Primer
cccgcgcccgccgcccaagtggatg

SEQ ID NO: 26 - DNA, 26 bp, Artificial Sequence
  Primer
tggggaacatgggtcggcatcactac

SEQ ID NO: 27 - DNA, 26 bp, Artificial Sequence
  Primer
gtagtgatgccgacccatgttcccca

SEQ ID NO: 28 - DNA, 25 bp, Artificial Sequence
  Primer
gcatgccctgtgtaggaggcccgac

SEQ ID NO: 29 - DNA, 25 bp, Artificial Sequence
  Primer
gtcgggcctcctacacagggcatgc

SEQ ID NO: 30 - DNA, 26 bp, Artificial Sequence
  Primer
tggtcgggcctctcacacagggcatg

SEQ ID NO: 31 - DNA, 25 bp, Artificial Sequence
  Primer
atcgaaggatcccgggcggccttcc

SEQ ID NO: 32 - DNA, 25 bp, Artificial Sequence
  Primer
ggaaggccgcccgggatccttcgat

SEQ ID NO: 33 - DNA, 25 bp, Artificial Sequence
  Primer
gccctggaccggtggttgcgcatag

SEQ ID NO: 34 - DNA, 25 bp, Artificial Sequence
  Primer
ctatgcgcaaccaccggtccagggc

SEQ ID NO: 35 - DNA, 27 bp, Artificial Sequence
  Primer
cacaacccagccaacgatgccttactg

SEQ ID NO: 36 - DNA, 27 bp, Artificial Sequence
  Primer
cagtaaggcatcgttggctgggttgtg

SEQ ID NO: 37 - DNA, 26 bp, Artificial Sequence
  Primer
ccatgaagtcaaggggcacgcagctg

SEQ ID NO: 38 - DNA, 26 bp, Artificial Sequence
  Primer
cagctgcgtgccccttgacttcatgg

SEQ ID NO: 39 - DNA, 28 bp, Artificial Sequence
  Primer
gatatgaccgtctttaatttcctcatgg

SEQ ID NO: 40 - DNA, 28 bp, Artificial Sequence
  Primer
ccatgaggaaattaaagacggtcatatc

SEQ ID NO: 41 - protein, 21 aa, Artificial Sequence
  Synthetic peptide
RKRDDSHQSDESHHSDESDEL

SEQ ID NO: 42 - protein, 21 aa, Artificial Sequence
  Synthetic peptide
RKRDDAHQADEAHHADEADEL

SEQ ID NO: 43 - protein, 21 aa, Artificial Sequence
  Synthetic peptide
RKRDDSHQADESHHADEADEL

SEQ ID NO: 44 - protein, 21 aa, Artificial Sequence
  Synthetic peptide
RKRDDAHQSDEAHHSDESDEL

SEQ ID NO: 45 - DNA, 25 bp, Artificial Sequence
  Primer
catccacttgggcggcgggcgcggg

SEQ ID NO: 46 - DNA, 26 bp, Artificial Sequence
  Primer
catgccctgtgtgagaggcccgacca

SEQ ID NO: 47 - protein, 584 aa, Homo sapiens
MKMMLVRRFRVLILMVFLVACALHIALDLLPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASAR

SEQ ID NO: 48 - protein, 271 aa, Homo sapiens
RGGCGRSSNRLARFADGTRACVRYGINPEQIQGEALSYYLARLLGLQRHVPPLALARVEARGAQWAQVQEELRAAHWTEGSVVSLTRWLPNLTDVVVPAPWRSEDGRLRPLRDAGGELANLSQAELVDLVQWTDLILFDYLTANFDRLVSNLFSLQWDPRVMQRATSNLHRGPGGALVFLDNEAGLVHGYRVAGMWDKYNEPLLQSVCVFRERTARRVLELHRGQDAAARLLRLYRRHEPRFPELAALADPHAQLLQRRLDFLAKHILHCK

SEQ ID NO: 49 - protein, 263 aa, Xenopus tropicalis
RGCGRSTNRMATLSDGSRVCVRYGINPEQIQGELLSYYLSRLLGLRGVPPCVLSRVGSPQWAPVQTELAATGWTQGALVSFTPWLHNLSSVLPPMALRAQDGKLRPLRTELGKGGATMLELAQWADLILFDYLTANFDRLVSNLFSLQWDPHIMLRGTNNLHRTPDGALVLLDNEAGFAHGYRLLGTWDKYNEQLLGTVCVFRRSVVRKVREMYLARNFAQELQALYLQEEPLAAELGLLSEAQAQTLQGRVGHEYKHMLSCQ

SEQ ID NO: 50 - protein, 263 aa, Danio rerio
AGCGRPTNRLASFADGTRACVRYGIDADQVLGETLSYYLAELLGISNLPPLSLSKLNLSSEQWASVRENIEVLEWSPNAIVSLTEFIPNVTGVFIPAHLQKQQQLSAESLSNMTLSYLLELMQWGDLILFDYLTANFDRIVSHIFSLQWDARVMERTTNNLLKTVNGDLLFIDNEAGLVHGYRVLDLWERYHKILLSSSCMFRRSTVRRVSELKRSGNSAKLLYELYRAREPLARELGSLSEEHALTLQSRIDVVYKHIKLCT

SEQ ID NO: 51 - protein, 268 aa, Branchiostoma floridae
QEGCGRMLNRLLSLEDGSLSCARYRQNTDQIQGEVFSYYLSRVLEIPNVPPCVLSTVNTNSHQWAEVSHDVHLSQWNEGKLVILSRWADHLIPAYIPRKLRYDGRRLHPVPEDLEGLSAAELGELAQWSDLIVFDYLTANFDRVANNMFNRQWNSQIMDSPVHNLEKSATDGSLVFFDNESGLLHSYRLLDKYAHFHDSLLKSLCVFRKRTAHIIESLHKKKDVGDLLLKEFKANEPLQRWLPFLPDANLRTLNERIDSVYEQIHKCR

SEQ ID NO: 52 - protein, 316 aa, Drosophila melanogaster
EQGCGRMQNRMVVFADGTRACARYRQNTDQIQGEIFSYYLGQLLNISNLAPSAATVVDTSTPNWAAALGDITQAQWKERRPVVLTRWLSDLEPAGIPQPFQPLERHLNKHDVWNLTRHMQSERQAQSQPHGLLKRLGAASSPGSAHQSNAIEETGTGTETANGALVQRLIELAQWSDLIVFDYLIANLDRVVNNLYNFQWNADIMAAPAHNLARQSASQLLVFLDNESGLLHGYRLLKKYEAYHSLLLDNLCVFRRPTIDALRRLRAAGAGRRLRDLFERTTSAGVRDVLPSLPDKSVKILVERIDRVLGQVQKCQ

SEQ ID NO: 53 - protein, 274 aa, Pediculus humanus
KEGCGRMQNRLLKFSDGTISCCRYRRNTDQIQGELFSYYLGLTLGLRNLVPTTLGLVKMRDTKWSSVRSEMMYAQWVEDKPVVLTKFVPNLITAFIPFNFRNSNRRLHPTDVSYNTSQVTGDILDNLVELAQWSDMIIFDYLTANLDRVVNNLYNLQWNFAMMDAPTHNLRRTIHDKLLIFLDNESGLLHGYRLLDKYESYHSALLNSLCVFRKSTAEIIKNLWKNGNVGDILLNKFNENEPDFKDYLPSLPEKSIQILNERITKVYNIIEKCE

SEQ ID NO: 54 - protein, 266 aa, Ixodes scapularis
REGCGRMQNRLVVFESGTLSCARYRQNNDQIQGELFSFYLARELGIANLPPAVLTRAAGRSWDSVRSNLELAQWRSDRPLVLTKFVDDLVPTNIPVEFRSPAARRLHPPDVFNKTDADIRELVQWSDLIIFDYLTANLDRIVNNMFNLQWNPEMMNSPAHNLLRQKSTGLLVFLDNESGLVHGYRLLDKYEVYHRSLLDSLCVFRKATAEAVARLSRDRDIGRRLFSAFNISDPGMDNYLPFLPERSVKILNKRLAQVNSQVRRCK

SEQ ID NO: 55 - protein, 290 aa, Homo sapien
VGYKGTQLKALLILEGGQKVVFKPKRYSRDHVVEGEPYAGYDRHNAEVAAFHLDRILGFHRAPLVVGRFVNLRTEIKPVATEQLLSTFLTVGNNTCFYGKCYYCRETEPACADGDIMEGSVTLWLPDVWPLQKHRHPWGRTYREGKLARWEYDESYCDAVKKTSPYDSGPRLLDIIDTAVFDYLIGNADRHHYESFQDDEGASMLILLDNAKSFGNPSLDERSILAPLYQCCIIRVSTWNRLNYLKNGVLKSALKSAMAHDPISPVLSDPHLDAVDQRLLSVLATVKQCT

SEQ ID NO: 56 - protein, 291 aa, Branchiostoma floridae
VGYKGTQLKALLVLNGHQKAVFKPKRYPREYIIEGKPYDGYDRHNAEIAAFHLDRLLGFRRAPLVVGRKVDLRTEIMPVGSERLMSTFLTQGNNTCFYGKCYYCKETEPACADGEVMEGSVTLWLPSNWSLKGRQRHPWGRTYRNDKQARWEYDDTYCNSVKQTPPYSSGPRLLDILDAAIFDYLIGNADRHHYETFEKDGDKGMLLLLDNAKSFGNPNYDERTILAPIYQCCKLRSSTWERMKLLTGDRLSQLLQKSLSHDPIAPILSDAHLAAMDRRLLTTIDMVQGCI

SEQ ID NO: 57 - protein, 290 aa, Strongylocentrotus purpuratus
VGFKGTQLKARLMLEGGQLVVFKPKRYPRDHVIYGTPYAGYDRHNGEIAAFHLDRVLDFRRAPLVVGRTLNLKTEILPVASPALNDTFLEKNSNICFYGKCYYCKPEEAACADGFTMEGSVTLWLPPDLKLKKWRHLWSRTYKDGKKAKWETDDQYCVNLMTRVPPEQHPLVLHMTDGAVFDYLIGNADRHMYETFEKDGDRGMLLHMDNAKSFGNPYLDEGTILAPIYQCCRLRRSTWNTLQQFKDGRLSQVMGQVLSHDPIAPVLTIWHLEALDRRLNDIIDTMHNCF

SEQ ID NO: 58 - protein, 292 aa, Nematostella vectensis
VGYKGTQLKATLLLEGNQKVVFKPGRYSREHVIKGKPYDGYDRHNGEIAAFHLDRVLGFNRAPPVAGRVVDLQDEIEPIAERNLLHTFFKKGGNTCFYGVCYYCNKEEAACANKTSMEGSMTIWLPQGWALRKWRHPWQRTYNNRKASWELDNNHCKKVIQQSPYDQGPRLLDIIDTAVFDFLIGNADRHHYETFKKGDDEGMLVHLDNAKSFGNPDHDELSIAAPLYQCCQLRDSTYKRLKEIANNKVKPVGELLKEATSSDALAPVLTEPHFKAVTRRLSIVMDIVTRCI

SEQ ID NO: 59 - protein, 292 aa, Ciona intestinalis
ALPKGTQIKLLLVLEGGQRVVFKQKRYARDHIIEGKPYDGYDRHNAEIAAFHLDRILDYRRSPLVVGRVVNLKTEIMPVATTRLLDTFREKDGNICYFGVCLYCNEKNMACGDGDVIEGSVTLWLPEKWSVFEKLRHPYQRTYVDNRMARWEKDETYCEKVVKHQPPYDRGTRLLDVADAAVFDYLIGNADRHHYEIFKNKSKDAMLLMLDNAKSFGNPSHHEPSILAPLRQCCILRNSTWIKLQKLSGGVLTQLLEAAMANDPISPVLHPSHLNAMDVRLPTLIETMEKCI

SEQ ID NO: 60 - protein, 286 aa, Drosophila melanogaster
NLGRGTQLKLLVRLSHQQKVIFKPQWYPREEVIDGMIYSGKDRHTAEVYAFYLGAVLDFRWTPIVVGRVVNLKKEIYAKGDPELQQTINIETDEDGREKYCLFGKCHYCNEEETVCGDERHNIEGVLIYIVPGTLAKRRSPWQRTYKDDKRAPWEDDMTYCKSLKNKMETIRLLDLIDASIFDYLIQNGDRHHYETREERVVLIDNGKAFGNPNKDHLDILAPLYQCCLLRKSTWDRLQVFSGGVLTEIIDRLSKQDALYPLITDKHKKGVERRLLVVYAVVEHCM

SEQ ID NO: 61 - protein, 288 aa, Pediculus humanus
NAKKGTQLKLMLTLEGGQQALFKPQWYPRNEIITGPVYSGRDRHNGEVAAFYLSLLLNMRTVPITIGKKINLRSEIMPYATPELLDTFYDDGNNTCFYGVCLYCEPKSLVCAKNDIMEGALIMWLPTKIKFKKYPSPWQRSYKSNLVARWEMDENYCSDVKNSKQNNQNRLLDLTDAAIFDFLIDNGDRHHYEVPLGFNHSSIFLFDNGKSFGNPYIDHIDILAPLYQCCVIRKSTWERLKMFSGGLLSSSLRNLLKYSDITPVLTNSHLFALDRRQLFIFAAVEMCI

SEQ ID NO: 62 - protein, 297 aa, Apis mellifera
QKEGGTQLKLQINYSNMQALFKPMRFPREQQTLPNHFYFTDFERHTAEIAAFHLDRLLGFRRAMPVTGRTLNLTTEIYQIADGELLKTFFVSPAGNICFHGKCSYYCDTAHAICGNPDTLEGSFAAFLPDKSFVARKAWRHPWRRSYHKRKKAQWEHDSDYCSLVKEIPPYHEGRRLLDLMDMAVLDFLMGNMDRHHYETFKIFGNNTFPLHLDHGRGFGRPFHDEISILAPILQCCMIRQTTLSTLLKFHNGPVPLSEALRKSMAKDPVAPVLWEPHLAALDRRVRVILQAIRDCV

SEQ ID NO: 63 - protein, 298 aa, Pediculus humanus
QKEGGTQLKLIIDYKNTMQALFKPMRFPREQQTLPNHFYFTDFERHNAEIAAFHLDRLLDFRRAMPVTGRKLNMTSELYALAEGELLKTFFVSPSNNLCFHGKCSYYCDTAHAVCGNPDMLEGSFAAFLPDTDLVPRKVWRHPWRRSYHKRRKAQWELDPNYCAAVREVEPYDRGRRLLDLMDMAVLDFLMGNMDRHHYETFRIFGNDTFPIHLDHGRGFGKPFHDEISILAPILQCCLLRRSTLSTLLKYHNGPVKLSDAMREAMKSDPVSPILWEPHFAALDRRIRIILEGIRDCI

SEQ ID NO: 64 - protein, 298 aa, Drosophila melanogaster
QKEGGTQLKLIIEYPNDIKALMKPMRFPREQQTLPNHFYFTDYERHNAEIAAFHLDRILGFRRAMPVAGRTLNITTEIYQLAEENLLKTFFVSPSLNLCFHGKCSYYCDTSHAICGNPDMLEGSFAAFLPNFESGNRKLWRHPWRRSYHKRKKAQWETDANYCALVRDIPPYDDGRRLYDLMDMAVFDFLTGNMDRHHYETFKVYGNETFPLHLDHGRGFGRPFHDELSILAPVLQCCLIRKSTLVKLLDFHNGPKPLSQLMSESLSQDPVSPVLWQPHLEALDRRTGIILQSIRDCI

SEQ ID NO: 65 - protein, 299 aa, Strongylocentrotus purpuratus
EKSGGTQVKMVLTFRDGGQSLMKPWRVQREYETLPDHFYFSDIERHNAEISAFHLDRILDFRRAPPVAGRWFNITKDLFELADPAFRKTFFRSPANNVCFVGHCSYYCETETAVCGKPDMIEGSVAAFLPSFKSAPRKTWRHPWRRSYSKHRTAVWEQDPTYCHRIVMNKHPYNSGRRLLDVMDMSIFDFLMGNMDRHHYETFEAFGNFTFPIHLDHGRAFGKHHHDELSILAPLIQCCRLRQSTHERVKLLASDRYRLSDVMRDSLATDRLAPVIIEEHLEALDRRLSIILEQLTRCV

SEQ ID NO: 66 - protein, 298 aa, Saccoglossus kowalevskii
EKSGGTQVKLIIDFIDGGQALFKPWKVPRDYQTVPDHFYFSDIERHNAEIASFHLDRILDFRRAPPQVGRWVNLTSQIYDLADSTLRRSFFRSPANNVCFVGHCSYYCQTETAVCGHPDMIEGSFAAYLPPFKMAPRKTWRHPWRRSYSKHRKAIWEDDPNYCQDVRNKHPYNTGRRLLDLMDLAVFDFLIGNMDRHHYETFSKFGNYTYPLHLDQGRAFGKYAHDELSILVPIVHCCVIRKSTHTRLQLLATSNFQLSDVMRDSMSTDKIAPVVFEPHLEALDRRLGIILKTVDNCI

SEQ ID NO: 67 - protein, 299 aa, Homo sapiens
VKPSGVHLKLVLRFSDFGKAMFKPMRQQRDEETPVDFFYFIDFQRHNAEIAAFHLDRILDFRRVPPTVGRIVNVTKEILEVTKNEILQSVFFVSPASNVCFFAKCPYMCKTEYAVCGKPHLLEGSLSAFLPSLNLAPRLSVPNPWIRSYTLAGKEEWEVNPLYCDTVKQIYPYNNSQRLLNVIDMAIFDFLIGNMDRHHYEMFTKFGDETFIIHLDNGRGFGKYSHDELSILVPLQQCCRIRKSTYLRLQLLAKEEYKLSLLMAESLRGDQVAPVLYQPHLEALDRRLRVVLKAVRDCV

SEQ ID NO: 68 - protein, 299 aa, Danio rerio
VKPSGLHLKLALKLQDFGKAMFKPMRQERHEETPENFFYFVDFQRHNAEIAAFHLDRVLDFRRVPPVAGRFVNVTSEILYITHNEELRSVFFTSPANNTCFFAKCLYVCKSEYAVCGHPDLLEGSMSAYLPGLSIAPRISIPNPWIRSYSFTGTEEWEVNPSYCDTVRKHYPYNSGNRLLNMIDMAVFDFLTGNMDRHHYEIFTKFGDEGFLLHLDNARGFGRHSHDELSILAPLTQCCMIKRSTLFRLKLLSSSEYLLSDVMRESLSRDALSPVLTEEHLQALDRRLKHTLLAVDTCV

SEQ ID NO: 69 - protein, 299 aa, Homo sapiens
MKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCV

SEQ ID NO: 70 - protein, 299 aa, Xenopus tropicalis
MKTGGTQLKLILTFQNYGQALFKPMKQTREQETPPDFFYFSDYERHNSEIAAFHLDRILDFRRVPPVSGRLVNMTKEIRDITRDKTLLRTFFISPANNICFYGECSYYCSTEHALCGKPDQLEGSLAAFLPDLSLAKRKNWRNPWRRSYHKRKKAEWEVDPNYCEGVKMTPPYDSGTRLLDLIDMTVFDFLMGNMDRHHYETFEKFGNETFIIHLDNGRGFGKYTHDELSILVPLIQCCRIKKSTYLRLQLLAKEEYLLSRVMEESLQLDKLAPLLYRPHLDALDRRLRIVLNAVSDCL

SEQ ID NO: 71 - protein, 299 aa, Danio rerio
MKSGGTQLKLIMSFQNYGQALFKPMKQTREQETPPDFFYFSDFERHNAEIAAFHLDRILDFRRVPPVAGRLVNMTREIRDVTRDKKLWRTFFVSPANNICFYGECSYYCSTEHALCGKPDQIEGSLAAFLPDLALAKRKTWRNPWRRSYHKRKKAEWEVDPDYCDEVKQTPPYDRGTRLLDIMDMTIFDFLMGNMDRHHYETFEKFGNDTFIIHLDNGRGFGKHSHDEMSILVPLTQCCRVKRSTYLRLQLLAKEEYKLSSLMEESLLQDRLVPVLIKPHLEALDRRLRLVLKVLSDCV

SEQ ID NO: 72 - protein, 300 aa, Caenorhabditis elegans
IMDGGTQVKFVFTFKNDKQAVFKPMRFGRDYESDPNHFYFSDFERHHAEIATFHLDRVLGFRRAIPTVGRVLNMTTELFEKAEKKLKKTFFFSPAKNFCFVSRCDYYCDTTHAICGLPDMKEGSVQVFLPDESAVPRKHNRSPYRRTYSKKNQVAEWQSSMNYCTDKVKTKRQYAHGRRLLDLVDIHILDYLIGNQDRHHFESFNVFNDPSYAIHLDHGRAFGRSDFDDDDIILPLRQCCILRPSTFQTLMNFYSTPKSLTKALHESLSKDPAHPILAYKHYPAMERRLAKIMSHILECF